SEQUENCE LISTING (14 sequences)

SEQ ID NO: 1 - DNA, 99 bp, Artificial Sequence
  primer
cgaggatccggccatggagcctcccggccgccgcgagtgtccctttccttcctggcgctttcctgggttgcttctggcggccatggtgttgctgctgta

SEQ ID NO: 2 - DNA, 69 bp, Artificial Sequence
  primer
ctccttctccgatgcctgtgagcatcatcatcatcatcatgagccaccaacatttgaagctatggagct

SEQ ID NO: 3 - DNA, 58 bp, Artificial Sequence
  primer
caggaaggaaagggacactcgcggcggccgggaggctccatggccggatcctcgagct

SEQ ID NO: 4 - DNA, 70 bp, Artificial Sequence
  primer
atgctcacaggcatcggagaaggagtacagcagcaacaccatggccgccagaagcaacccaggaaagcgc

SEQ ID NO: 5 - DNA, 40 bp, Artificial Sequence
  primer
ccatagcttcaaatgttggtggctcatgatgatgatgatg

SEQ ID NO: 6 - protein, 335 aa, Homo sapiens
CEEPPTFEAMELIGKPKPYYEIGERVDYKCKKGYFYIPPLATHTICDRNHTWLPVSDDACYRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKGPRPTYKPPVSNYPGYPKPEEGILDSLDVWVIAVIVIAIVVGVAVICVVPYRYLQRRKKKGKADGGAEYATYQTKSTTPAEQRG

SEQ ID NO: 7 - protein, 341 aa, Homo sapiens
CEHHHHHHEPPTFEAMELIGKPKPYYEIGERVDYKCKKGYFYIPPLATHTICDRNHTWLPVSDDACYRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKGPRPTYKPPVSNYPGYPKPEEGILDSLDVWVIAVIVIAIVVGVAVICVVPYRYLQRRKKKGKADGGAEYATYQTKSTTPAEQRG

SEQ ID NO: 8 - protein, 495 aa, Artificial Sequence
  vector
KFTIVFPHNQKGNWKNVPSNYHYCPSSSDLNWHNDLIGTGLQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNDICPTVHNSTTWHSDYKVKGLCDSNLISTDITFFSEDRELSSLGKEGTGFRSNYFAYETGDKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSGLHLSSKAQVFEHPHIQDAASQLPDDEILFFGDTGLSKNPIDFVEGWFSSWKSSIASFFFIIGLIIGLFLVLRVGIYLYIKLKHTKKRQIYTDIEMNRLGR

SEQ ID NO: 9 - protein, 501 aa, Artificial Sequence
  vector
KHHHHHHFTIVFPHNQKGNWKNVPSNYHYCPSSSDLNWHNDLIGTGLQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNDICPTVHNSTTWHSDYKVKGLCDSNLISTDITFFSEDRELSSLGKEGTGFRSNYFAYETGDKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSGLHLSSKAQVFEHPHIQDAASQLPDDEILFFGDTGLSKNPIDFVEGWFSSWKSSIASFFFIIGLIIGLFLVLRVGIYLYIKLKHTKKRQIYTDIEMNRLGR

SEQ ID NO: 10 - protein, 501 aa, Artificial Sequence
  vector
KFTIVFPHNQKGNWKNVPSNYHYCPSSSDLNWHNDLIGTGLQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNDICPTVHNSTTWHSDYKVKGLCDSNLISTDITFFHHHHHHSEDRELSSLGKEGTGFRSNYFAYETGDKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSGLHLSSKAQVFEHPHIQDAASQLPDDEILFFGDTGLSKNPIDFVEGWFSSWKSSIASFFFIIGLIIGLFLVLRVGIYLYIKLKHTKKRQIYTDIEMNRLGR

SEQ ID NO: 11 - protein, 598 aa, Artificial Sequence
  vector
MAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 12 - protein, 604 aa, Artificial Sequence
  vector
MHHHHHHAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 13 - protein, 598 aa, Artificial Sequence; identical to SEQ ID NO: 11
  vector

SEQ ID NO: 14 - protein, 604 aa, Artificial Sequence; identical to SEQ ID NO: 12
  vector

(19) United States
(12) Patent Application Publication
(10) Pub. No.: US 2005/0158712 A1
(43) Pub. Date: Jul. 21, 2005

(54) Methods for purifying viral particles for gene therapy

(75) Inventors: Philippe Leboulch, Charlestown, MA (US); Sergei Aleshkov, Kenmore, NY (US)

Correspondence Address: LAHIVE & COCKFIELD, LLP., 28 STATE STREET, BOSTON, MA, 02109, US

(73) Assignee: Genetix Pharmaceuticals, Inc., Cambridge, MA (US)

(21) Appl. No.: 11/016,560

(22) Filed: Dec. 17, 2004

Related U.S. Application Data
(63) Continuation of application No. PCT/US03/19612, filed on Jun. 20, 2003.
(60) Provisional application No. 60/390,461, filed on Jun. 21, 2002.

(51) Int. Cl. C12Q 1/70; C07H 21/04; C12N 7/02; C07K 14/005

(52) U.S. Cl. 435/5; 435/239; 530/350

(57) ABSTRACT

Novel methods of purifying and concentrating viral particles are disclosed for use in gene therapy, vaccines and viral standards preparation and other possible applications involving preparation and purification of viral particles. The viral particles are purified after the addition of a peptide tag to a protein on the surface of the viral particle, e.g., the envelope, coat or cellular membrane proteins. The viral particles are isolated by affinity absorption specific for the peptide tags. Also disclosed are methods of using the isolated viral particles in gene therapy.

### RELATED APPLICATIONS

The present application is a continuation of PCT/US03/19612, filed on Jun. 20, 2003 and U.S. provisional patent application Ser. No. 60/390,461, filed on Jun. 21, 2002, which are expressly incorporated by reference.

### BACKGROUND OF THE INVENTION

Efficient purification of functional viral particles is a crucial step in development of gene therapy vectors, vaccines and viral standards preparation, etc. The development of efficient gene-transfer techniques has led to important progress toward human gene therapy. The early development of the field focused on a technique called ex vivo gene therapy in which autologous cells are genetically manipulated in culture prior to transplantation. Recent advances have stimulated the development of in vivo gene therapy approaches based on direct delivery of the therapeutic genes to cells in vivo. The rate-limiting technologies of gene therapy are the gene delivery vehicles, called vectors.

The most efficient vectors are based on recombinant versions of viruses. Viruses are obligate intra-cellular parasites designed through the course of evolution to infect cells, often with great specificity to a particular cell type. Viruses tend to be very efficient at transfecting their own DNA into the host cell, which is expressed to produce new viral particles. By replacing genes that are needed for the replication phase of their life cycle (the non-essential genes) with foreign genes of interest, the recombinant viral vectors can transduce the cell type they would normally infect.

Though a number of viruses have been developed, retroviral (including lentiviral) vectors serve as prototypes in gene therapy. Retroviruses are RNA viruses that reverse transcribe their genome upon infection of a susceptible cell. This double-stranded DNA form of the virus is capable of being integrated into the chromosome of the infected cell, the viral DNA genome is integrated as a single copy into essentially random sites within the host genome. Following integration, the viral genome replicates along with the host genome, guaranteeing its passage to all progeny cells. These distinguishing features make retroviruses excellent vectors for stable gene transfer.

The production of high titer and a large volume of virus is essential for in vivo gene therapy. However, means to concentrate and purify recombinant viruses are currently largely limited to physical separation (e.g., ultracentrifugation, gel filtration, chromatography, non-specific absorption). These traditional purification methods have drawbacks, notably, co-purification of contaminants (which can be toxic to target cells), extended purification times and the ability to process only limited volumes.

Significant effort has been directed towards development of improved methods for obtaining high viral titers. The relatively large size and fragile structure of viruses, however, has rendered this difficult. Accordingly, to realize the true potential of gene therapy, a significant need exists in the art to develop substantially improved techniques for the purification and concentration of viral vectors.

### SUMMARY OF THE INVENTION

The present invention provides improved methods for isolating viral particles. This is achieved by adding a peptide tag to a protein on the surface of the viral particle, and then isolating (e.g., purifying and/or concentrating), the viral particle by affinity absorption specific for the peptide tag. The peptide tag can be added to the surface protein using any suitable technique, such as chemical linking or genetic co-expression. Accordingly, the peptide can be added directly to a surface protein on the virus or can be added separately to the protein, followed by adding the tagged protein to the surface of the viral particle. In addition, in situations where it is advantageous to subsequently remove the peptide tag from the surface protein (e.g., following purification), the peptide tag can include one or more specific protease cleavage sites.

In a particular embodiment of the invention, the surface protein is a viral envelope protein, such as VSV-G. In a preferred embodiment, the tagged VSV-G protein comprises the nucleotide sequence shown in SEQ ID NO:9 or SEQ ID NO:10. In another particular embodiment of the invention, the surface protein is a viral coat protein, such as VP2 or VP3. In a preferred embodiment, the tagged VP2 protein comprises the nucleotide sequence shown in SEQ ID NO:12. In another preferred embodiment, the tagged VP3 protein comprises the nucleotide sequence shown in SEQ ID NO:14. In yet another particular embodiment of the invention, the surface protein is a cellular membrane protein, e.g., a transmembrane protein, such as a GP anchored protein or CD46. In a preferred embodiment, the tagged CD46 protein comprises the nucleotide sequence shown in SEQ ID NO:7.

Accordingly, in one aspect, the present invention provides a method for purifying viral particles comprising expressing a peptide tag together with a protein on the surface of the viral particle, and isolating the viral particle by affinity absorption specific for the peptide tag. In another aspect, the invention provides a method for purifying viral particles comprising adding a tagged surface protein (e.g., an envelope protein or a cellular membrane protein) to naked virions or packaging cells producing naked virions and isolating the virions by affinity absorption specific for the peptide tag. The tagged surface protein can be produced separately from the naked virion by, for example, chemically linking the peptide tag to the surface protein or by recombinantly expressing the tag and the protein together as a single fusion protein, and then added to (e.g., by mixing or co-incubation) the naked virion or cells producing the naked virion.

In other embodiments, the present invention includes tagged surface proteins which can be employed in the foregoing methods, as well as viral particles produced by the foregoing methods. The viral particles can be produced by, for example, transiently transfecting eukaryotic packaging cells with a nucleic acid (e.g., DNA vector) encoding the tagged surface protein. Alternatively, the viral particles can be produced by co-expressing the peptide tag and the protein in eukaryotic packaging cells after chromosomal integration of a nucleic acid (e.g., DNA) encoding the tagged protein.

Any of a variety of art recognized peptide tags can be employed in the present invention. For example, suitable peptide tags include a: FLAG peptide; short FLAG peptide; His-6 peptide; Glutathion-S-Transferase (GST); Staphylococcal protein A; Streptococcal protein G; Calmodulin; Calmodulin binding peptides; Thioredoxin; β-galactosidase; Ubiquitin; Chloramphenicol cetyltransferasel S-peptide (Ribonuclease A, residues 1-20); Myosin heavy chain; DsbA; Biotin subunit; Avidin; Streptavidin; Strp-tag; c-Myc; Dihydrofolate reductase; CKS; Polyarginine; Polycisteine; Polyphenylalanine; lac Repressor; N-terminus of the growth hormone; Maltose binding protein; Galactose binding protein; Cyclomaltodextrin glucanotransferase; Callulose binding domain; Haemolysin A; TrpE or TrpLE; Protein kinase sites; BAI epitope; Btag; VP7 region of Bluetongue virus; and Green Fluorescent Protein. In a preferred embodiment, the peptide tag is a Histidine-6 tag.

Similarly, a variety of art recognized affinity absorption techniques can be employed in the present invention, including any technique which uses the specific interaction which occurs between a peptide tag its ligand or substrate. Suitable affinity absorption techniques include, for example, techniques which rely on the specific interaction that occurs between an enzyme and it's substrate, or an antigen and an antibody. Preferred affinity absorption techniques include affinity chromatography, affinity precipitation, sedimentation with affinity resin of magnetic beads, and immunoassays.

Accordingly, affinity absorption techniques used in the present invention include those which employ moieties specific for the aforementioned peptide tags, such as nickel; cobalt; anti-FLAG monoclonal antibodies; nitrilotriacetic acid; glutathione-sepharose; IgG-sepharose; Albumin; Organic and peptide ligands, DEAE-sephadex; Calmodulin; ThioBond™ resin; TPEG-sepharose; Chloramphenicol-sepharose; S-protein (ribonuclease A, residues 21-124); Biotin; Strptaviding Anti-myc antibody; Methotrexate agarose; S-sepharose; Phenyl-superose; lac Operator; Amylose resin; Galactose-sepharose; α-Cyclodextrin-agarose; Cellulose; and Anti-BTag antibodies.

The methods of the present invention can be used to isolate any viral particle having or capable of having a protein on its surface, including a variety of retroviral and lentiviral particles. Particular viruses include, but are not limited to MoMSV; HaMuSV; MuMTV; GaLV; FLV; spumavirus; Friend; MSCV; RSV; HTLV-1; HTLV-2; HIV-1; HIV-2; SIV; FIV; and EIV. The viral particles can further include an exogenous gene desired for delivery to a cell, such as a therapeutic gene for treating a disease (e.g., to be employed in gene therapy). The viral particles can also include other well known genes and genetic regulatory elements required or advantageous for gene therapy, such as a marker gene (e.g., GFP) to help trace integration of the viral particle into the genome of the cell.

In certain embodiments where addition of a peptide tag to the viral surface protein disrupts the normal function of the protein, a mix of both tagged and untagged forms of the surface protein can be used. In addition, tagged and/or untagged surface proteins which are pseudotyped envelope proteins can be used, in addition to or in place of the viral particles natural envelope protein. Accordingly, in another embodiment, the present invention provides a method for purifying viral particles by selectively adding a protein tag to certain surface proteins and not to others, and/or by adding a mixture of tagged and untagged surface proteins to a viral particle, such as a naked viral particle or packaging cells producing naked viral particles, and then isolating the viral particles by affinity absorption specific for the peptide tag. This allows for efficient isolation of the viral particle without disrupting the function of the surface protein.

For delivery to cells, viral particles of the present invention are preferably used in conjunction with a suitable packaging cell line or co-transfected into cells in vitro along with other vector plasmids containing the necessary retroviral genes (e.g., gag and pol) to form replication incompetent virions capable of packaging the vectors of the present invention and infecting cells.

Accordingly, in yet another embodiment, the invention provides a method of delivering a gene to a cell (which is then integrated into the genome of the cell) by contacting the cell with a viral particle according to the present invention. The cell (e.g., in the form of tissue or an organ) can be contacted (e.g., infected) with the viral particle (virion) ex vivo and then delivered to a subject (e.g., a mammal, animal or human) in which the gene will be expressed. Alternatively, the cell can be contacted with the virion in vivo by, for example, administering the virion to a subject or a localized area of a subject (e.g., localized vasculature). The cell can be autologous to the subject (i.e., from the subject) or it can be non-autologous (i.e., allogeneic or xenogenic) to the subject. Moreover, the viral particles of the present invention are capable of being delivered to both dividing and non-dividing cells. Thus, the cells can be from a wide variety including, for example, bone marrow cells, mesenchymal stem cells (e.g., obtained from adipose tissue), synovial fibroblasts, chondrocytes and other primary cells derived from human and animal sources.

Accordingly, the present invention provides substantially improved methods and compositions for use in gene therapy, vaccines and viral standards preparation and other possible applications involving preparation and purification of viral particles, as well as substantially improved methods for producing and isolating viral particles.

### DETAILED DESCRIPTION OF THE INVENTION

Unlike previously described methods for purifying viral particles, the present invention provides an improved method for isolating viral particles more efficiently and with greater purity. In addition, in certain embodiments, viral vectors isolated according to the present invention have an increased capacity to infect cells, thereby making them more useful in methods of gene therapy.

According to the methods of the present invention, viral particles are purified by adding a peptide tag to a protein on the surface of the viral particle, and then isolating the viral particle by affinity absorption specific for the peptidic tag. The peptide tag can be added to any protein on the surface of the viral particle, such as an envelope protein, a coat protein or a cellular membrane protein. Typically, the peptide tag is expressed together with the protein on the surface of the viral particle, although it can also be chemically linked to the protein or added to the protein separately from the viral particle.

Any suitable peptide tag and corresponding ligand and/or substrate can be used in the affinity absorption techniques of the present invention, as are well known in the art. In a preferred embodiment, the affinity absorption is based on resin of magnetic beads bearing moieties specific for a particular peptide tag. In another preferred embodiment, the affinity absorption is based on affinity column chromatography bearing moieties specific for a particular peptide tag. These methods of isolating viral particles have an intrinsic advantage over the physical separation purification methods of the prior art in that they provide specific and rapid purification without disrupting the large and fragile structure of viral particles. In addition, viral particles isolated in this manner can be purified and concentrated without the need for centrifugation.

Prior to the present invention, viruses were previously thought to be unamenable to purification using peptide tags and affinity purification techniques due to their delicate structure and composition. The present invention shows, for the first time, how this can be efficiently achieved without detriment to the virus or its function.

Definitions

As used herein, the following terms and phrases used to describe the invention shall have the meanings provided below.

The terms “virus,” “virion” and “viral particle” are used interchangeably, and include all viruses (e.g., enveloped and non-enveloped) which express proteins on their surface, including envelope proteins, coat proteins and cellular membrane proteins, as well as “naked’ viruses which lack such surface proteins but which can be modified to include them (e.g., by insertion of the proteins into the outer lipid bilayer of the virus). Such viruses include for example, but are not limited to, retroviruses (which include type C retroviruses, lentiviruses and spumaviruses) and adenoviruses.

Retroviruses are a class of enveloped viruses containing a single stranded RNA molecule as the genome. Following infection, the viral genome is reverse transcribed into double stranded DNA, which integrates into the host genome and is expresses as proteins, The viral genome is approximately 10 kilobases, containing at least three genes: gag (coding for core proteins), pol (coding for reverse transcriptase) and env (coding for viral envelope protein). At each end of the genome are long terminal repeats (LTRs) which include promoter/enhancer regions and sequences involved with integration. In addition, there are sequences required for packaging the viral DNA (psi) and RNA splice sites in the env gene.

Accordingly, the term “retrovirus” refers to any known retrovirus (e.g., type c retroviruses, such as Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend, Murine Stem Cell Virus (MSCV) and Rous Sarcoma Virus (RSV)). “Retroviruses” of the invention also include human T cell leukemia viruses, HTLV-1 and HTLV-2 viruses.

Generally, a requirement for retroviral integration and expression of viral genes is that the target cells should be dividing. This limits gene therapy to proliferating cells in vivo or ex vivo. However, lentiviruses are a subclass of retroviruses which are able to infect both proliferating and non-proliferating cells and are thus also encompassed by the present invention. Thus, “retroviruses” of the invention also include the lentiviral family of retroviruses, such as human Immunodeficiency viruses, HIV-1, HIV-2, simian immunodeficiency virus (SIV), feline immunodeficiency virus (FIV), equine immunodeficiency virus (EIV), and other classes of retroviruses.

The term “adenovirus” refers to non-enveloped viruses containing a linear double stranded DNA genome. The life cycle of adenoviruses does not normally involve integration into the host genome, rather they replicate as episomal elements in the nucleus of the host cell.

Other viruses which can be employed (e.g., produced and/or isolated) in the present invention include alphaviruses such as Eastern Equine Encephalomyelitis virus (EEEV), Western Equine Encephalomyelitis virus (WEEV), Venezuelan Encephalomyelitis virus (VEV), Sindbis virus, Semliki Forest virus (SFV) and Ross River virus (RRV), the rhinoviruses such as human rhinovirus 2 (HRV2) and human rhinovirus type 89 (HRV89), the polioviruses such as poliovirus 2 (Pv2) and poliovirus 3 (PV3), simian virus 40 (SV40), viruses from the tobacco mosaic virus group such as Tobacco Mosaic virus (TMV), Cowpea Mosaic virus (CMV) Alfalfa Mosaic virus (AmV), Cucumber Green Mottle Mosaic virus watermelon strain (CGMMV-W) and Oat Mosaic virus (OMV) and viruses from the brome mosaic virus group such as Brome Mosaic virus (BMV), broad bean mottle virus and cowpea chlorotic mottle virus. Additional suitable viruses include Rice Necrosis virus (RNV), adenovirus type 2 and geminiviruses such as tomato golden mosaic virus (TGMV), cassaya latent virus and maize streak virus. Additional viruses which may be suitable include hordeivirus, ilarvirus, luluvirus, tombuvirus, potexvirus, luteovirus, carmovirus, tymovirus, sobemovirus, tobravirus, furovirus, and dianthvirus.

The term “vector” refers to a nucleic acid molecule capable of transporting another nucleic acid to which it has been linked. The term “expression vector” includes any vector, (e.g., a plasmid, cosmid or phage chromosome) containing a gene construct in a form suitable for expression by a cell (e.g., linked to a promoter). In the present specification, “plasmid” and “vector” are used interchangeably, as a plasmid is a commonly used form of vector. Moreover, the invention is intended to include other vectors which serve equivalent functions.

The term “viral vector” refers to a vector containing structural and functional genetic elements that are primarily derived from viruses as defined herein, e.g., retroviral vectors (which include type C retroviral vectors, lentiviral vectors and spumaviral vectors), adenoviral vectors, adenovirus-associated viral vectors, SV40 vectors, Semliki Forest virus vectors, Sindbis vectors, etc., as well as other vectors which serve equivalent functions. Viral vectors employed in the present invention can be transfected into, for example, “packaging cell lines” which refer to cell lines (typically mammalian cell lines) which contain the necessary coding sequences to produce viral particles which lack the ability to package RNA and produce replication-competent helper-virus. When the packaging function is provided within the cell line (e.g., in trans by way of a plasmid vector), the packaging cell line produces recombinant virus, thereby becoming a “viral producer cell line.” Accordingly, viral particles of the present invention can be isolated from packaging cell supernatants.

Viral particles which can be isolated by the methods of the present invention include a broad variety of viruses. For example, the virus can be an “enveloped virus” which are a class of viruses whose core is surrounded by the viral envelope. The viral envelope is usually a lipid bilayer produced upon budding from the packaging cell's plasma membrane and also comprises one or more proteins encoded by viral genes referred to herein as “viral envelope proteins.” The term “viral envelope protein” refers to a protein in the viral envelope which interacts with a specific cellular protein to determine the target cell range of the virus. “Viral envelope proteins” include both naturally occurring (i.e., native) envelope proteins and functional derivatives thereof, as well as synthetic forms thereof (e.g., recombinantly produced viral envelope proteins).

As is well known in the art, altering the viral envelope (env) gene or its gene product can be used to manipulate the target cell range of the virus. For example, replacing the env gene of one virus with the env gene of another virus (referred to as “pseudotyping”) can extend the host range of a virus. Thus, a “pseudotyped virus” refers to a virus having an envelope protein that is from a virus other than the virus from which the viral genome is derived. For example, the envelope protein can be from a retrovirus of a species different from the retrovirus from which the RNA viral genome is derived or from a non-retroviral virus (e.g., vesciular stomatitis virus or “VSV”).

The present invention also can be used to isolate “non-enveloped” viruses. Non-enveloped viruses have an external structure primarily composed of a “viral coat protein” encoded by viral genes. Accordingly, as used herein, the term “viral coat protein” refers to proteins which create the tightly assembled structure of the protective shell for non-enveloped viruses and prevent degradation of the genome by environmental factors.

In addition, the present invention can be used to isolate “naked virions”. As used herein, the term “naked virion” refers to virions produced by membrane budding, e.g., from packaging cells, in the absence of expressed envelope protein. However, naked virions contain cell-specific proteins in the lipid membrane referred to herein as “cellular membrane proteins.” As used herein, the term “semi-synthetic viral vectors” refers to a viral particle produced by adding a separately produced recombinant envelope protein, with or without pseudotyping, to a naked virion.

The terms “transformation” and “transfection” refer to the introduction of a nucleic acid, e.g., an expression vector, into a recipient cell. Transfection or transformation may be accomplished by a variety of means known in the art including but not limited to calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “transduction” refers to the delivery of a gene(s) using a viral or retroviral vector by means of viral infection rather than by transfection. In preferred embodiments, retroviral vectors are transduced by packaging the vectors into virions prior to contact with a cell. For example, an anti-HIV gene carried by a retroviral vector can be transduced into a cell through infection and provirus integration.

The term “transgene” means a nucleic acid sequence (e.g., a therapeutic gene), which is partly or entirely heterologous, i.e., foreign, to a cell into which it is introduced, or, is homologous to an endogenous gene of the cell into which it is introduced, but which is designed to be inserted into the genome of the cell in such a way as to alter the genome (e.g., it is inserted at a location which differs from that of the natural gene or its insertion results in “a knockout”). A transgene can include one or more transcriptional regulatory sequences and any other nucleic acid, such as introns, that may be necessary for optimal expression of a selected nucleic acid.

As used herein, the term “affinity absorption” refers to any method that utilizes the specific interaction which occurs between a peptide tag its ligand or substrate. For example, “affinity absorption” includes methods which use the specific interaction that occurs between an enzyme and its substrate or an antigen and an antibody. Such methods are exploited in a variety of art recognized techniques, such as “affinity chromatography,” “affinity precipitation,” “sedimentation with affinity resin of magnetic beads” and “immunoassays” to isolate, i.e., purify and concentrate, the viral particles.

Recombinant Viral Vectors

Recombinant viral vectors can be made using a variety of art recognized techniques. Suitable sources for obtaining viral (e.g., retroviral) sequences for use in forming the vectors include, for example, genomic RNA and cDNAs available from commercially available sources, including the Type Culture Collection (ATCC), Rockville, Md. The sequences also can be synthesized chemically.

Any suitable expression vector can be employed for generating the viral vectors of the present invention. Suitable expression constructs include human cytomegalovirus (CMV) immediate early promoter constructs. The cytomegalovirus promoter can be obtained from any suitable source. For example, the complete cytomegalovirus enhancer-promoter can be derived from the human cytomegalovirus (hCMV). Other suitable sources for obtaining CMV promoters include commercial sources, such as Clontech, Invitrogen and Stratagene. Part or all of the CMV promoter can be used in the present invention. Other examples of constructs which can be used to practice the invention include constructs that use MuLV, SV40, Rous Sarcoma Virus (RSV), vaccinia P7.5, PGK, EF-1-alpha and rat β-actin promoters. In some cases, such as the RSV and MuLV, these promoter-enhancer elements are located within or adjacent to the LTR sequences.

Suitable regulatory sequences required for gene transcription, translation, processing and secretion are art-recognized, and are selected to direct expression of the desired protein in an appropriate cell. Accordingly, the term “regulatory sequence”, as used herein, includes any genetic element present 5′ (upstream) or 3′ (downstream) of the translated region of a gene and which control or affect expression of the gene, such as enhancer and promoter sequences. Such regulatory sequences are discussed, for example, in Goeddel, Gene expression Technology: Methods in Enzymology, page 185, Academic Press, San Diego, Calif. (1990), and can be selected by those of ordinary skill in the art for use in the present invention.

In one embodiment, the invention employs an inducible promoter within the retroviral vectors, so that transcription of selected genes can be turned on and off. This minimizes cellular toxicity caused by expression of cytotoxic viral proteins, increasing the stability of the packaging cells containing the vectors. For example, high levels of expression of VSV-G (envelope protein) and Vpr can be cytotoxic (Yee, J.-K., et al., Proc. Natl. Acad. Sci., 91: 9654-9568 (1994) and, therefore, expression of these proteins in packaging cells used in connection with vectors of the invention can be controlled by an inducible operator system, such as the inducible Tet operator system (GIBCOBRL), allowing for tight regulation of gene expression (i.e., generation of retroviral particles) by the concentration of tetracycline in the culture medium. That is, with the Tet operator system, in the presence of tetracycline, the tetracycline is bound to the Tet transactivator fusion protein (tTA), preventing binding of tTA to the Tet operator sequences and allowing expression of the gene under control of the Tet operator sequences (Gossen et al. (1992) PNAS 89: 5547-5551), In the absence of tetracycline, the tTA binds to the Tet operator sequences preventing expression of the gene under control of the Tet operator.

Examples of other inducible operator systems which can be used for controlled expression of the protein which provides a pseudotyped envelope are 1) inducible eukaryotic promoters responsive to metal ions (e.g., the metallothionein promoter), glucocorticoid hormones and 2) the LacSwitch™ Inducible Mammalian Expression System (Stratagene) of E. coli. Briefly, in the E. coli lactose operon, the Lac repressor binds as a homotetramer to the lac operator, blocking transcription of the lac2 gene. Inducers such as allolactose (a physiologic inducer) or isopropyl-β-D-thiogalactoside (IPTG, a synthetic inducer) bind to the Lac repressor, causing a conformational change and effectively decreasing the affinity of the repressor for the operator. When the repressor is removed from the operator, transcription from the lactose operon resumes.

In yet another approach, selective expression of retroviral genes contained within the viral vectors of the invention can be achieved by cloning in a Cre/lox repressor system upstream of selected coding sequences. Specifically, a polystop signal can be inserted between the gene(s) to be selectively expressed and a 5′ promoter. The polystop signal is flanked by two loxP1 sites (Sauer (1993) Methods in Enzymology 225: 890-900). Upon contact with cre recombinase, the lox sites will recombine and delete the polystop signal, allowing the promoter to act in cis to turn on expression of the gene(s).

Peptide Tags, Tagging and Isolation of Viral Particles

Peptide Tags

As used herein, the term “peptide tag” refers to a peptide sequence which is added to a protein on the surface of a viral particle, or to a protein which can be attached to the surface of a viral particle, to facilitate purification of the viral particle.

Peptide tags can be added to any surface protein, such as an envelope protein, a coat protein or a cellular membrane protein. Typically, the peptide tag is expressed together, in the proper reading frame, with the protein on the surface of the viral particle. The peptide tag also can be covalently or non-covalently linked to the surface protein using, for example, a variety of well known chemical linkages and linking reagents. The peptide tag also can be added directly to the viral particle or separately from the viral particle and then attached to the viral particle. The peptide tag can further include one or more protease cleavage sites for subsequent removal of the peptide tag from the viral particle.

Accordingly, as used herein, the term “tagged protein” or “tagged surface protein” refers to any protein on the surface of a viral particle, or capable of being added or attached to the surface of a viral particle, which includes one or more peptide tags or sequences as defined above. As previously described, the peptide tag can be linked, e.g., genetically, covalently or otherwise, to the viral surface protein thereby forming a hybrid or “tagged” protein. Moreover, if the peptide tag disrupts the normal function of the surface protein, then a mixture of tagged and untagged surface proteins can be used, either of the same protein or different proteins having the same function. For example, a mixture of tagged and untagged forms of the same envelope protein can be used, or a mixture of a tagged form of an envelope protein and an untagged form of a different envelope protein (e.g., a pseudotyped envelope protein) can be used so as to have at least one functioning envelope protein. This can be achieved by, for example, selectively adding (or expressing) the tag only to certain surface proteins, by adding (or expressing) a mixture of tagged and untagged proteins to the viral particle, by adding (or expressing) tagged proteins to a viral particle already containing or expressing untagged proteins, or by adding (or expressing) untagged proteins to a viral particle already containing or expressing tagged proteins.

A broad variety of art-recognized peptide tags can be employed in the present invention. For example, suitable peptide tags include, but are not limited to: FLAG peptide; short FLAG peptide; His-6 peptide; Glutathion-S-Transferase (GST); Staphylococcal protein A; Streptococcal protein G; Calmodulin; Calmodulin binding peptides; Thioredoxin; β-galactosidase; Ubiquitin; Chloramphenicol acetyltransferasel S-peptide (Ribonuclease A, residues 1-20); Myosin heavy chain; DsbA; Biotin subunit; Avidin; Streptavidin; Strp-tag; c-Myc; Dihydrofolate reductase; CKS; Polyarginine; Polycisteine; Polyphenylalanine; lac Repressor; N-terminus of the growth hormone; Maltose binding protein; Galactose binding protein; Cyclomaltodextrin glucanotransferase; Callulose binding domain; Haemolysin A; TrpE or TrpLE; Protein kinase sites; BAI eptiope; Btag; VP7 region of Bluetongue virus; and Green Flourescent Protein.

The foregoing exemplary peptide tags are described in further detail below.

FLAG™ Binding Peptide Tag

The FLAG epitope was originally described as consisting of a highly charged and therefore soluble eight amino acid peptide (DYKDDDDK) that is recognized by commercially available monoclonal antibodies M1 and M2 raised against this peptide. The M1 antibody binds this peptide in a calcium dependent manner. The fusion of this peptide sequence into the vectors of interest allows for purification using an anti-FLAG affinity column. In one embodiment, the FLAG peptide can be incorporated into, for example, a coat protein of a non-enveloped virus, an envelope protein of an enveloped virus, or an integral cellular membrane protein of an enveloped virus, using standard protocols for site directed mutagenesis. In another embodiment, only four amino acids of the FLAG peptide (DYKD), the “short FLAG” is sufficient for purification using an anti-FLAG affinity column.

In one embodiment, the virus is purified with, for example, phosphorylcholine-Sepharose affinity chromatography. In another embodiment, the extracts containing virus expressing the FLAG peptide (e.g., the FLAG peptide or the short FLAG) are purified by affinity chromatography using the anti-FLAG M1 and the anti-FLAG M2 affinity columns. Using the FLAG tag as the affinity handle, an anti-FLAG-M1 affinity gel (Eastman Kodak Company, New Haven, Conn., USA) can be used. In a particular embodiment, before loading onto the column, the fraction containing the viruses is dialyzed against TBS and filter sterilized. The chromatography is carried out, for example, at 4° C. or according to the instructions of the manufacturer. The column is washed, for example, three times with 5 mL of TBS. Bound vectors are eluted by adding glycine-HCl buffer and immediately neutralized.

Histidine-Six (His-6) Peptidic Tag

His-6 tags consist of six histidine residues linked or fused to the protein of interest. The His-6 tag does not disrupt the protein structure and thus does not usually require removal following purification of the protein. The 6-His residues have a significant affinity for matrixes containing nickel and, thus, His-6-tagged proteins can be purified by, for example, binding to nickel ions on the matrix. Elution of the protein is accomplished under mild conditions by either reducing the pH or adding imidazole as a competitor. Other art-recognized protocols for using His-6 tags in affinity absorption techniques are also encompassed by the invention.

Glutathione S-Transferase (GST) Tag

GST tags can be added to proteins using a variety of well known techniques. In one embodiment, the pLEF vector (Rudert et al. (1996) Gene 169: 281-282.) can be used to genetically co-express the GST sequence with a the viral surface protein (e.g., as a fusion protein). The vector contains nucleotides encoding the GST tag and can be engineered also to express the surface protein together with the GST tag. The resulting viral particles containing the GST tagged surface protein can then be batch purified using, for example, GSH sepharose beads. Alternatively, oligohisitidine tailing of the tagged surface proteins can be performed, followed by purification using, for example, chromatography on nickel chelate affinity columns.

Calmodulin Binding Peptide (CBP) Tag

CBP tags can be added to viral surface proteins using a variety of well known techniques. In one embodiment, expression vectors, e.g., pCAL expression vectors, containing a sequence encoding a calmodulin binding peptide, are used. The CBP tag allows the hybrid tagged surface protein to bind to a calmodulin resin in the presence of low concentrations of calcium. Elution can be accomplished by, e.g., the presence of 2 mM EGTA under neutral pH conditions.

Streptococcal Protein G (SPG) Tag

Streptococcal protein G (SPG) binds with high affinity to serum albumin. SPG binds with serum albumin from various species, with highest affinity for serum albumin from rats, humans and mice. Accordingly, in one embodiment, the albumin binding domains B2A3 (BA) and/or B I A2B2A3 (BABA) from SPG are added to viral surface proteins, such as a coat protein of a non-enveloped virus, an envelope protein of an enveloped virus, or an integral cellular membrane protein, using the techniques described herein. Medium containing SPG tagged viruses can then be concentrated on, for example, S-Sepharose columns (Pharmacia, Piscataway, N.J.). The bound protein can then be eluted and purified by affinity chromatography using, for example, a polyclonal or monoclonal anti-BA or an anti-BABA antibody coupled to an affigel column (BioRad).

Tagging

As used herein, “tagging” refers to the addition or linking of a “peptide tag” to a protein on the surface of a viral particle, or a protein capable of being added or attached to the surface of a viral particle. As previously described, the peptide tag can be covalently or noncovalently linked to the protein, or it can be genetically co-expressed (fused) with the protein. Such tagging can be accomplished using, for example, standard site directed mutagenesis. Tagging also can be achieved by inserting or engineering the peptide tag onto a protein on the surface of a viral particle. Tagging can further include adding specific protease sites around the peptide tags to facilitate their subsequent cleavage and removal from the protein.

In a particular embodiment of the invention, the tagged protein on the surface of the viral vector is an envelope protein. In a preferred embodiment, the envelope protein is VSV-G. In another particular embodiment, the tagged protein on the surface of the viral particle is a viral coat protein. In a preferred embodiment, the coat protein is VP2. In another preferred embodiment, the coat protein is VP3. In yet another particular embodiment, the tagged protein on the surface of the viral particle is an integral cellular membrane protein. In a preferred embodiment, the cellular membrane protein is, for example, a transmembrane protein, a GP anchored protein, or CD46. In another preferred embodiment, the peptide tag added to a protein on the surface of a viral particle comprises the nucleic acid sequence shown in SEQ ID NO:7, 9, 10 or 12.

In other preferred embodiments, the peptide tag can be incorporated into, for example, a coat protein of a non-enveloped virus, an envelope protein of an enveloped virus, or an integral cellular membrane protein of an enveloped virus. In another embodiment, naked virions are tagged by tagging integral cellular membrane proteins on the surface of the naked virions. In a preferred embodiment, a tagged or untagged envelope protein is added to the tagged naked virions. In another preferred embodiment, the envelope protein is pseudotyped. In yet another embodiment, the naked virions with the tagged cellular membrane protein on the surface of the virion, are isolated by affinity absorption, and a free recombinant or synthetic viral envelope protein is added to the tagged naked virion. In one embodiment, the viral envelope is pseudotyped.

In a particular embodiment, free recombinant surface (e.g., envelope or cellular membrane) protein or an equivalent synthetic surface protein is tagged and added to naked virions or to packaging cells producing naked virions. The naked virions can be already tagged or can be untagged. In a particular embodiment, the method further comprises adding a mixture of both tagged and untagged proteins to the naked virion, with or without pseudotyping.

Vectors encoding tagged surface protein can be transiently transfected into eukaryotic packaging cells to produce tagged viral particles. Alternatively, the tagged surface protein can be expressed in eukaryotic packaging cells after stable chromosomal integration.

Isolation

As used herein, the term “isolation” refers to partial or complete removal of viral particles from the media in which they are produced. Isolation can be achieved using a variety of techniques for purifying and/or concentrating viral particles. The tagged viral particles can be purified by affinity absorption specific for the peptidic tag on the viral particle. As used herein, the term “affinity absorption” is intended to include any method which utilizes the specific interaction which occurs between a peptidic tag used in the present invention and its ligand or substrate. For example, “affinity absorption” can include methods which utilize the specific interaction which occurs between an enzyme and it's substrate or an antigen and an antibody, and which can be exploited in techniques such as “affinity chromatography,” “affinity precipitation,” “sedimentation using affinity resin of magnetic beads” and “immunoassays” to isolate, i.e., purify and concentrate the tagged viral vectors.

In a preferred embodiment, “affinity absorption” is achieved by affinity chromatography which is a chromatographic technique that depends on the specific affinity of one molecule for another. For example, enzymes may be isolated by binding an analogue of their normal substrate to an inert matrix. If a solution of mixed proteins is passed through a column packed with such a matrix, the required enzyme will be retained or retarded because of its affinity for the bound substrate. The protein is then retrieved by eluting the column using a suitable solution with a pH or ionic concentration such that the binding affinity is reduced.

For example, prepared virus containing conditioned medium can be collected from cell monolayers and the viral titer is determined. After filtration through 0.4 mkm membrane and special pre-treatment, the conditioned medium is applied on an affinity chromatography column which is packed with nickel-chelate resin (which binds to the His-6 peptide tag). The recombinant virions are eventually bound through their six histidine residue tags with immobilized nickel. After washing, the virus is eluted with gradient of the concentration of imidazol (5 mM-0.5 M) in the buffer containing 20 mM Tris/HCl, pH 7.4, 0.1 mM NaCl. Virus containing fractions were dialized against PBS and the viral titer was determined.

In another preferred embodiment, “affinity absorption” is achieved using sedimentation with the affinity resin. For example, prepared virus containing conditioned medium can be mixed with nickel-chelate resin on a rotation platform. After several washes the resin can be sedimented using low speed centrifugation and bound virus is eluted by resuspension with buffer containing 20 mM Tris/HCl, pH 7.4 0.1 M imidazol. Supernatant can then be cleared by additional round of centrifugation and the virus was dialized against PBS and the viral titer can be determined.

In yet another embodiment, “affinity absorption” is achieved using magnetic beads. For example, virus containing conditioned medium can be mixed with a suspension of magnetic beads with attached nickel ligand. After 8 hours of incubation on a shaker at 4° C., the suspension can be placed on a magnetic separator for 1 minute and the supernatant can be removed. Following three successive washes with PBS-5 mM imidiazol, the suspension can be mixed with elution buffer so that the final concentration of imidiazol is 0.1 M. The suspension can then be incubated for 5 minutes and placed on a magnetic separator and the eluate can be collected and dialyzed against PBS, pH 7.4 and the viral titer can be determined.

Various ligands and/or substrate specific for the peptide tags of the invention are known in the art and can be used. Peptide tag specific ligands and substrates encompassed by the present invention include, but are not limited to, anti-FLAG monoclonal antibodies; nitrilotriacetic acid; glutathione-sepharose; IgG-sepharose; Albumin; Organic and peptide ligands, DEAE-sephadex; Calmodulin; ThioBond™ resin; TPEG-sepharose; Chloramphenicol-sepharose; S-protein (ribonuclease A, residues 21-124); Biotin; Strptavidingl Anti-myc antibody; Methotrexate agarose; S-sepharose; Phenyl-superose; lac Operator; Amylose resin; Galactose-sepharose; α-Cyclodextrin-agarose; Cellulose; Anti-BTag antibodies. Examples of peptide tags and their respective ligands or substrates for isolating viral particles through the affinity absorption techniques of the invention are listed in Table 1.

TABLE 1

| PEPTIDE TAG | LIGAND/SUBSTRATE |
|---|---|
| FLAG peptide; short | anti-FLAG monoclonal antibodies |
| FLAG peptide | |
| His-6 peptide | nitrilotriacetic acid |
| Glutathion-S-Transferase (GST) | glutathione-sepharose |
| Staphylococcal protein A | IgG-sepharose |
| Streptococcal protein G | Albumin |
| Calmodulin | Organic and peptide ligands, DEAE- |
| | sephadex |
| Calmodulin binding peptides | Calmodulin; |
| Thioredoxin | ThioBond ™ resin |
| β-galactosidase | TPEG-sepharose |
| Chloramphenicol acetyltransferase | Chloramphenicol-sepharose |
| S-peptide (Ribonuclease A, | S-protein (ribonuclease A, |
| residues 1-20) | residues 21-124); |
| Avidin | Biotin |
| Streptavidin | Biotin |
| Strp-tag | Strptavidin |
| c-Myc | Anti-myc antibody |
| Dihydrofolate reductase | Methotrexate-agarose |
| Polyarginine | S-sepharose |
| Polycisteine | Thiopropyl-sepharose |
| Polyphenylalanine | Phenyl-superose |
| lac Repressor | lac Operator |
| Maltose binding protein | Amylose resin |
| Galactose binding protein | Galactose-sepharose |
| Cyclomaltodextrin | Alpha-cyclodextrin-agarose |
| glucanotransferase | |
| Cellulose binding domain | Cellulose |
| Btag | Anti-Btag antibodies |
| Chitin binding domain | Chitin |

Viral Envelope Proteins and Pseudotyping

The viral envelope proteins (env) determine the range of host cells which can ultimately be infected and transformed by recombinant retroviruses generated from the cell lines. In the case of lentiviruses, such as HIV-1, HIV-2, SIV, FIV and EIV, the env proteins include gp41 and gp120.

Examples of retroviral-derived env genes which can be employed in the invention include, but are not limited to type C retroviral envelope proteins, such as those from Moloney murine leukemia virus (MoMuLV), Harvey murine sarcoma virus (HaMuSV), murine mammary tumor virus (MuMTV), gibbon ape leukemia virus (GaLV), and Rous Sarcoma Virus (RSV). Other viral env genes which can be used include, for example, env genes from immunodeficiency viruses (HIV-1, HIV-2, FIV, SIV and EIV), human T cell leukemia viruses (HTLV-1 and HTLV-3), and Vesicular stomatitis virus (VSV) (Protein G). When producing recombinant retroviruses of the invention (e.g., recombinant lentiviruses), the wild-type retroviral (e.g., lentiviral) env gene can be used, or can be substituted with any other viral env gene, such those listed above. Methods of pseudotyping recombinant viruses with envelope proteins from other viruses in this manner are well known in the art.

In one embodiment, the invention provides packaging cells which produce recombinant lentivirus (e.g., HIV, SIV, FIV, EIV) pseudotyped with the VSV-G glycoprotein. The VSV-G glycoprotein has a broad host range. Therefore, VSV-G pseudotyped retroviruses demonstrate a broad host range (pantropic) and are able to efficiently infect cells that are resistant to infection by ecotropic and amphotropic retroviruses. (Yee et al. (1004) PNAS 91: 9564-9568. Any suitable serotype (e.g., Indiana, New Jersey, Chandipura, Piry) and strain (e.g., VSV Indiana, San Juan) of VSV-G can be used in the present invention. The protein chosen to pseudotype the core virion determines the host range of the packaging cell line. VSV-G interacts with a specific phospholipid on the surface of mammalian cells (Schlegel, R., et al., Cell, 32: 639-646 (1983); Spuertzi, F., et al., J. Gen. Virol., 68: 387-399 (1987)). Thus, packaging cell lines which utilize VSV-G to provide a pseudotyped envelope for the retroviral core virion have a broad host range (pantropic). Moreover, VSV-G pseudotyped retroviral particles can be concentrated more than 100-fold by ultracentrifugation (Burns, J. C., et al., Proc. Nat'l. Acad. Sci., 90: 8033-8037 (1993)). Stable VSV-G pseudotyped retrovirus packaging cell lines permit generation of large scale viral preparations (e.g. from 10 to 50 liters supernatant) to yield retroviral stocks in the range of 10⁷ to 10¹¹ retroviral particles per ml.

Viral envelope proteins of the invention (whether pseudotyped or not) can also be modified, for example, by amino acid insertions, deletions or mutations to produce targeted envelope sequences such as ecotropic envelope with the EPO ligand, synthetic and/or other hybrid envelopes; derivatives of the VSV-G glycoprotein. Furthermore, it has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein (Roux et al. (1989) PNAS 86: 9079-9083; Julan et al. (1992) J. Gen Virol 73: 3251-3255; and Goud et al. (1983) Virology 163: 251-254); or coupling cell surface receptor ligands to the viral env proteins (Neda et al. (1991) J Biol Chem 266: 14143-14146). Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, can also be used to convert an ecotropic vector in to an amphotropic vector.

Packaging Cell Lines

Any suitable packaging system (cell line) can be employed with the vectors of the present invention to facilitate transduction of host cells with the vectors in gene therapy. Generally, the packaging cells are mammalian cells, such as human cells. Suitable human cell lines which can be used include, for example, 293 cells (Graham et al. (1977) J. Gen. Virol., 36: 59-72, tsa 201 cells (Heinzel et al. (1988) J. Virol, 62: 3738), and NIH3T3 cells (ATCC)). Other suitable packaging cell lines for use in the present invention include other human cell line derived (e.g., embryonic cell line derived) packaging cell lines and murine cell line derived packaging cell lines, such as Psi-2 cells (Mann et al. (1983) Cell, 33: 153-159; FLY (Cossett et al. (1993) Virol., 193: 385-395; BOSC 23 cells (Pear et al. (1993) PNAS 90: 8392-8396; PA317 cells (Miller et al. (1986) Molec. and Cell. Biol., 6: 2895-2902; Kat cell line (Finer et al. (1994) Blood, 83: 43-50; GP+E cells and GP+EM12 cells (Markowitz et al. (1988) J. Virol., 62: 1120-1124, and Psi Crip and Psi Cre cells (U.S. Pat. No. 5,449,614; Danos, O. and Mulligan et al. (1988) PNAS 85: 6460-6464). Packaging cell lines of the present invention can produce retroviral particles having a pantropic amphotropic or ecotropic host range. Preferred packaging cell lines produce retroviral particles, such as lentiviral particles (e.g., HIV-1, HIV-2 and SIV) capable of infecting dividing, as well as non-dividing cells.

The packaging cell line may also provide for the vector to affect the range of host cells capable of being infected by providing a particular envelope protein (e.g., by pseudotyping).

Cell Transfection and Screening

The viral particles of the present invention can be transfected or transduced into host cells and tested for infectivity using standard transfection/transduction techniques. Generally cells are incubated (i.e., cultured) with the vectors or virions containing the vectors in an appropriate medium under suitable transfection conditions, as is well known in the art.

Positive packaging cell transformants (i.e., cells which have taken up and integrated the retroviral vectors) can be screened for using a variety of selection markers which are well known in the art. For example, marker genes, such as green fluorescence protein (GFP), hygromycin resistance (Hyg), neomycin resistance (Neo) and β-galactosidase (β-gal) genes can be included in the vectors and assayed for using e.g., enzymatic activity or drug resistance assays. Alternatively, cells can be assayed for reverse transcriptase (RT) activity as described by Goff et al. (1981) J. Virol. 38: 239 as a measure of viral protein production. Cells can also be measured for production of viral titers as is known in the art.

Similar assays can be used to test for the production of unwanted, replication-competent helper virus. For example, marker genes, such as those described above, can be included in the “producer” vector containing the viral packaging sequence (Ψ) and LTRs. Following transient transfection of packaging cells with the producer vector, packaging cells can be subcultured with other non-packaging cells. These non-packaging cells will be infected with recombinant, replication-deficient retroviral vectors of the invention carrying the marker gene. However, because these non-packaging cells do not contain the genes necessary to produce viral particles (e.g., TAR region), they should not, in turn, be able to infect other cells when subcultured with these other cells. If these other cells are positive for the presence of the marker gene when subcultured with the non-packaging cells, then unwanted, replication-competent virus has been produced.

Accordingly, to test for the production of unwanted helper-virus, hybrid lentiviral vectors of the invention can be subcultured with a first cell line (e.g., NIH3T3 cells) which, in turn, is subcultured with a second cell line which is tested for the presence of a marker gene or RT activity indicating the presence of replication-competent helper retrovirus. Marker genes can be assayed for using e.g., FACS, staining and enzymatic activity assays, as is well known in the art.

Uses in Gene Therapy

The isolated viral particles of the present invention can be used to transfer selected genes into dividing as well as non-dividing cells including, but not limited to, cells of the skin, gastrointestinal tissue, cardiac tissue, and neuronal tissue. Techniques for transfer of selected genes into tissue or cells using viral vectors are well-established in the art. Genes for selection and transfer via viral vectors are also well known. One of skill can thus use these established techniques with the isolated viral vectors of the present invention to efficiently transfer selected genes to cells and mammals. The rapid and specific purification techniques of the present invention are particularly desirable for gene transfer in human therapy.

Suitable genes which can be delivered via the viral particles of the invention include any therapeutic gene. For example, genes involved in promoting angiogenesis to treat ischemia can be delivered, such as genes encoding soluble Interleukin-1 α Receptor Type I, Soluble Interleukin-1α Receptor Type II, Interleukin-1α Receptor Antagonist Protein (IRAP), Insulin-Like Growth Factor (IGF), Tissue Inhibitors of Matrix Metallo-Proteinases (TIMP)-1,-2,-3,-4, Bone Morphogenic Protein (BMP)-2 and -7, Indian Hedgehog, Sox-9, Interleukin-4, Transforming Growth Factor (TGF)-β, Superficial Zone Protein, Cartilage Growth and Differentiation Factors (CGDF), Bcl-2, Soluble Tumor Necrosis Factor (TNF)-α Receptor, Fibronectin and/or Fibronectin Fragments, Leukemia Inhibitory Factor (LIF), LIF binding protein (LBP), Interleukin-4, Interleukin-10, Interleukin-11, Interleukin-13, Hyaluronan Synthase, soluble TNF-α receptors 55 and 75, Insulin Growth Factor (IGF)-1, activators of plasminogen, urokinase plasminogen activator (uPA), parathyroid hormone-related protein (PTHrP), and platelet derived growth factor (PDGF)-AA -AB or -BB.

Cells can be transfected or transduced either in vivo or ex vivo and then returned to a subject (see e.g., U.S. Pat. No. 5,399,346). Thus, the cells can be autologous (e.g., a bone marrow cell, mesenchymal stem cell obtained from adipose tissue, a synovial fibroblast or a chondrocyte) or non-autologous (i.e., allogeneic or xenogenic), such as cells from a cell line or from primary cells derived from a human or animal source.

### EXAMPLES

### Example 1

### Tagging of Cellular Membrane Proteins

CD46 is a single chain type I transmembrane protein with an intracellular cytosolic tail, one transmembrane domain and a large extracellular part. Thus, CD46 is an example of a cellular membrane protein. The crystal structure of the extracellular part is known (Casasnovas J M et al., EMBO J., 18, 2911-22) and available from the NIH PDB database under the aronym “1 CKL”. Analysis of the crystal structure of CD46 demonstrates that first three N-terminal amino acids, i.e., cysteine (C), glutamic acid (E), and glutamic acid (E) are exposed to the environment and are, therefore, favorable sites for incorporation of the peptidic tag sequence.

A. Incorporation of a Peptidic Tag

In order to incorporate a His-6 peptide tag (a sequence of six histidines) into CD46, such that the final CD46-His6 mutant contained the N-terminal sequence CEHHHHHHEPPT instead of CEEPPT of the wild type CD46 protein, a peptide tag was inserted between the two glutamic acids (E) to guarantee efficient cleavage of the signal peptide. Thus the first two N-terminal amino acids of the mature protein, i.e., cysteine and glutamic acid were left intact. Any art-recognized peptide tag can be used.

B. Mutagenesis of cDNA

The mutagenesis of CD46 cDNA was performed by substitution of its 5′ sequence with chemically synthesized oligonucleotides in the following manner:

1. Substrate Preparation

The substrate, i.e., CD46 cDNA (SEQ ID NO:6) cloned in a pBS-SK vector, was cleaved with Sac1 restriction endonuclease and large fragment containing pBS-SK and most of the CD46 cDNA was purified using gel-electrophoresis.

2. Preparation of Oligonucleotides

The following oligonucleotides were prepared:

CD46HisXd

(SEQ ID NO: 1)

(5′CGAGGATCCGGCCATGGAGCCTCCCGGCCGCCGCGAGTGTCCCTTTC
CTTCCTGGCGCTTTCCTGGGTTGCTTCTGGCGGCCATGGTGTTGCTGCTG
TA3′)
CD46His0db

(SEQ ID NO: 2)

(5′PhosCTCCTTCTCCGATGCCTGTGAGCATCATCATCATCATCATGAG
CCACCAACATTTGAAGCTATGGAGCT3′)
CD46HisXr

(SEQ ID NO: 3)

(5′PhosCAGGAAGGAAAGGGACACTCGCGGCGGCCGGGAGGCTCCATGG
CCGGATCCTCGAGCT3′)
CD46His0ra

(SEQ ID NO: 4)

(5′ATGCTCACAGGCATCGGAGAAGGAGTACAGCAGCAACACCATGGCCG
CCAGAAGCAACCCAGGAAAGCGC3′)
CD46His0rb

(SEQ ID NO: 5)

(5′PhosCCATAGCTTCAAATGTTGGTGGCTCATGATGATGATGATG3′)

The five oligonucleotides were mixed in equimolar amounts at concentrations of 0.5 nM/μl and annealed by gradually decreasing the temperature from 98° C. to 4° C. for 3 hours.

3. DNA Ligation

The annealed oligonucleotides were mixed with Sac1 digested pSK-CD46cDNA and ligated using T4 DNA ligase for 1 hour at room temperature.

4. Cloning and Analysis

E. coli were transformed with the ligation mixture under standard conditions as recommended by the manufacturer (Invitrogen, Carlsbad, Calif.) and plated on 15% agar plates containing 100 μg/ml ampicillin. The resulting colonies were isolated and the DNA samples from their minipreps were analysed by digestion with Sac 1, Xho 1 and BamH1. The DNA structure of the mutated areas was further confirmed by DNA sequencing.

5. Construction of the Vectors for Expression of Recombinant CD46 and Cd46His6

Vectors pHCMV-G, pSK-CD46 and pSK-CD46His6 were digested with Xho1 restriction endonuclease and pHCMV-G Xho1 digest was additionally treated with calf intestine alkaline phosphatase (CIP). All three linear DNAs were isolated and purified using gel-electrophoresis. Two ligation mixtures containing equimolar amounts of Xho I linearized plasmids were prepared in the following manner:
a) pHCMV-G and pSK-CD46, and
b) pHCMV-G and pSK-CD46His6.

Ligations were performed with T4 DNA ligase. E. coli were transformed with the ligation mixtures under standard conditions as recommended by the manufacturer (Invitrogen, Carlsbad, Calif.) and plated on 15% agar plates containing 100 μg/ml ampicillin. The resulting colonies were isolated and the DNA samples from their minipreps were analysed by digestion with Sac1, Xho1 and BamH1.

Correct expression vectors pHCMV-CD46 and pHCMV-CD46His6 contain cDNAs of CD46 (SEQ ID NO: 6) and CD46His6 (SEQ ID NO:7) under control of the immediate early promoter of human cytomegalovirus followed by the second rabbit β-globin intron and rabbit β-globin polyadenylation signal.

### Example 2

### Tagging of Envelope Proteins

The spike protein of vesicular stomatitis virus (VSV-G) is a virus-encoded transmembrane glycoprotein which consists of a cytoplasmic tail, a transmembrane domain and a large ectodomain. Thus, VSV-G is an example of a virus-specific envelope protein.

A. Incorporation of the Peptide Tag

To incorporate a His-6 tag peptide tag into VSV-G, the His6 tag was incorporated between the first amino acid residue, i.e., lysine, of mature VSV-G and the second amino acid residue of the processed VSV-G, i.e., phenylalanine. Thus, the first positively charged amino acid residue of the mature protein, which is necessary for efficient cleavage of the signal peptide, was preserved. In the alternative, the N-terminal amino acid residues of the VSV-G can be exposed to the environment and, therefore, can also be used as sites for insertion of the peptide tag.

B. Mutagenesis of cDNA

The mutagenesis of VSV-G cDNA, including substrate preparation, preparation of oligonucleotides, ligation, cloning and analysis and construction of vectors for expression of wild-type VSV-G (SEQ ID NO:8) and its polyhistidine mutants (SEQ ID NO: 9 and SEQ ID NO:10) was performed using the same methods as described in Example 1 above. The polyhistidine mutants shown in SEQ ID NO:9 and SEQ ID NO:10 were constructed to demonstrate that peptide tags can be incorporated into different, selected parts of a protein of interest. In addition, different tags can be incorporated into the same protein. For example, two, three or more peptide tags can be positioned in different parts of the same protein or virion. These tags can be the same (e.g., two, three or more polyhistidine tags), or they can be different (e.g., a mix of different tags such polyhistidine and calmodulin binding domain tags). This allows for the generation of a mix of different protein mutants.

### Example 3

### Tagging of Coat Proteins

Tagging of VP2 Coat Protein

The virus specific coat protein, VP2 (SEQ ID NO:11), which is an AAV (adeno-associated virus) specific coat protein was tagged as follows.

A. Incorporation of Peptide Tag

A His-6 tag peptide tag was incorporated into VP2 between the first and second amino acid residues of wild-type VP2.

B. Mutagenesis of cDNA

The mutagenesis of VP2 cDNA, including substrate preparation, preparation of oligonucleotides, ligation, cloning and analysis and construction of vectors for expression of wild-type VP2 (SEQ ID NO:11) and its polyhisitidine mutant (SEQ ID NO:12) was performed using the same methods as described in Example 1 above.

Tagging of VP3 Coat Protein

The virus specific coat protein, VP3 (SEQ ID NO:13), which is another AAV (adeno-associated virus) specific coat protein was tagged as follows.

A. Incorporation of Peptide Tag

A His-6 tag peptide tag was incorporated into VP3 at amino acid residue 587 of the wild-type VP3 protein. This site on the wild-type VP3 protein was chosen because it is efficiently exposed at the top of the structural loop in the mature AAV mature capsid. In addition, incorporation of exogenous peptide sequences at this site does not disrupt the biological, e.g., binding activities, of the wild-type VP3 protein.

B. Mutagenesis of cDNA The mutagenesis of VP3 cDNA, including substrate preparation, preparation of oligonucleotides, ligation, cloning and analysis and construction of vectors for expression of wild-type VP3 (SEQ ID NO:13) and its polyhisitidine mutant (SEQ ID NO:14) was performed using the same methods as described in Example 1 above.

### Example 4

### Isolation of Viral Particles

The tagged viral particles of the invention, including those described in Examples 1-3 above, can be isolated, e.g., purified and/or concentrated, using a variety of art-recognized affinity absorption techniques. For example, two principal approaches for purification and enrichment of the tagged viral particles of the invention through column affinity chromatography and sedimentation with the affinity resin of magnetic beads are exemplified below.

Both techniques were performed using a His-6 tag and its corresponding ligand, i.e., immobilized Nickel-chelate resin. It is understood in the art that different tags require different ligands. Such known tags and their respective ligands are encompassed by the present invention.

A. Affinity Column Chromatography

Prepared virus containing conditioned medium was collected from cell monolayers and the viral titer was determined. After filtration through 0.4 mkm membrane and special pre-treatment, the conditioned medium was applied on an affinity chromatography column which was packed with nickel-chelate resin. The recombinant virions were eventually bound through their six histidine residue tags with immobilized nickel. After washing, the virus was eluted with gradient of the concentration of imidazol (5 mM-0.3 M) in PBS, pH 7.4 and the viral titer was determined.

B. Sedimentation with the Affinity Resin

Prepared virus containing conditioned medium was mixed with nickel-chelate resin on a rotation platform. After several washes the resin was sedimented using low speed centrifugation and bound virus was eluted by resuspension with buffer containing PBS, 0.1 M imidiazol pH 7.4. Supernatant was cleared by additional round of centrifugation, the virus was dialized against PBS and the viral titer was determined.

C. Determination of Viral Titers

The viral titers of the tagged viral particles isolated, e.g., purified and/or concentrated, using the affinity absorption techniques of the invention can be determined by a variety of art-recognized means all of which are intended to be encompassed by the present invention.

In an exemplary method, viral titers were determined using eGFP fluorescence along with G-418 resistance of NIH 3T3 cells. The purification/concentration yields and viral titers for VSVG-His6 mutant pseudotyped with recombinant EGFP/Neo HIV 1 as taught by the methods of the present invention are summarized in Table 2.

TABLE 2

| | | | Protein | | | | |
|---|---|---|---|---|---|---|---|
| | Volume | % | Concentration | Titer | Total Virus | Fold | Fold |
| Sample | (ml) | Yield | (ug/ml) | (IU/ml) | (IU) | Purification | Concentration |
| Crude | 100 | 100 | 360 | 1.2 × 10⁶ | 1.2 × 10⁸ | 1 | 1 |
| Conditioned | | | | | | | |
| Medium | | | | | | | |
| Pooled Peak | 1.6 | 96 | 80 | 7.2 × 10⁷ | 1.15 × 10⁸ | 270 | 62.5 |
| Fractions | | | | | | | |

Equivalents

Those skilled in the art will recognize, or be able to ascertain using no more than routine experimentation, many equivalents to the specific embodiments of the invention described herein. Such equivalents are intended to be encompassed by the following claims. The entire contents of all references, patents and published patent applications cited throughout this application are hereby incorporated by reference.

1. A method for purifying viral particles comprising:
a. adding a peptide tag to a protein on the surface of the viral particle, and
b. isolating the viral particle by affinity absorption specific for the peptide tag.

2. The method of claim 1, wherein the peptide tag is added to the protein by chemical linking.

3. The method of claim 1, wherein the peptide tag is added to the protein by genetic co-expression.

4. The method of claim 1, wherein the peptide tag comprises a protease cleavage site.

5. The method of claim 1, wherein the protein is an envelope protein.

6. The method of claim 5, wherein the envelope protein is VSV-G.

7. The method of claim 1, wherein the tagged protein comprises the nucleotide sequence shown in SEQ ID NO:9 or SEQ ID NO:10.

8. The method of claim 1, wherein the protein is a viral coat protein.

9. The method of claim 8, wherein the viral coat protein is VP2 or VP3.

10. The method of claim 1, wherein the tagged protein comprises the nucleotide acid sequence shown in SEQ ID NO:12 or SEQ ID NO:14.

11. The method of claim 1, wherein the protein is a cellular membrane protein.

12. The method of claim 1 1, wherein the cellular membrane protein is selected from the group consisting of a transmembrane protein, a GP anchored protein, and CD46.

13. The method of claim 1, wherein the peptide tag comprises the nucleotide sequence shown in SEQ ID NO:7.

14. The method of claim 1, further comprising the step of transiently transfecting a eukaryotic packaging cell line with a nucleic acid encoding the tagged protein.

15. The method of claim 1, wherein the peptide tag and the protein are co-expressed in eukaryotic packaging cells after chromosomal integration of DNA encoding the peptide tag and the protein.

16. A method for purifying viral particles comprising:
a. expressing a peptide tag together with a protein on the surface of the viral particle, and
b. isolating the viral particle by affinity absorption specific for the peptide tag.

17. A method for purifying viral particles comprising:
a. adding a tagged protein to naked virions or packaging cells producing naked virions; and
b. isolating the virions by affinity absorption specific for the peptidic tag.

18. The method of claim 17, further comprising adding an untagged protein to the naked virions.

19. A viral particle produced by the method of claim 1.

20. A viral particle having a surface protein comprising a peptide tag.

21. A viral particle having a surface protein comprising an amino acid sequence selected from the group consisting of SEQ ID NOS:7, 9, 10 and 12.
